(19) United States
(12) Patent Application Publication
(10) Pub. No.: US 2007/0065395 A1
(43) Pub. Date: Mar. 22, 2007

(54) Topical compositions containing plant extracts for personal care

(76) Inventor: Jung Ja Kim, Seoul (KR)

Correspondence Address: ST. ONGE STEWARD JOHNSTON & REENS, LLC, 986 BEDFORD STREET, STAMFORD, CT, 06905-5619, US

(21) Appl. No.: 11/229,186

(22) Filed: Sep. 16, 2005

(51) Int. Cl. A61K 8/97; A61K 36/8962

(52) U.S. Cl. 424/74; 424/754

(57) ABSTRACT

A topical composition or a personal beauty care product (e.g., soap, shampoo, rinse, hair conditioner, and the like) comprises chemical or biological substances or plant extracts obtained from certain plants, in particular, plant species of the family Liliaceae or Amaryllidaceae, preferably selected from the following Allium species such as Allium fistulosum, Allium ampeloprasum or Allium porrum, or other Allium species or their relative species having ingredients which can stimulate hair growth and/or reduce hair loss. The plant species can preferably be selected from bunching onion, scallion, Welsh onion, green onion, great green onion, spring onion, leek, wild leek, Egyptian leek, shallot, and Korean or Asian great green onion, or the like.

### FIELD OF THE INVENTION

The present invention relates to topical compositions and hair and skin care products containing plant extracts or components usable for stimulating hair growth and reducing hair loss. More particularly, the present invention relates to topical compositions and hair/skin care products (e.g., soap, shampoos, rinses, hair and body conditioner, and the like) containing plant components obtained from certain plants, preferably from plant species of the family Liliaceae (e.g. bunching onion, scallion, Welsh or green onion, leek, shallot, and the like) which can stimulate hair growth or reduce hair loss while also functioning for hair or skin care products, such as cleaning and protecting skin or hair with natural substances.

### BACKGROUND OF THE INVENTION

Factors and causes contributing the loss of human hair, and solutions and treatments for preventing it and/or improving hair growth conditions to the scalp have been the subjects of research for long time, however, with limited success.

According to modern researches, common causes in the loss of hair or alopecia of varying degree are told to include postinfective defluvium and androgenic or other alopecias developed typically by traumas, chronic illness, medicines, hormonal imbalance, or the like. For example, violent traumas, such as accidents, surgery, severe fractures, can be followed by hair loss within several months. Such hair loss may be spontaneous and reversible over time. Alopecias may also be developed after prolonged treatment with certain medicine, for example, corticosteroids and cytostatic drugs that can cause blocking of the intense cellular mitotic activity of the hair bulb, occurring with relative frequency. The reversibilty of such alopecias depends typically on the damage suffered by the bulb cells as a result of the more or less prolonged use of the said drugs.

Several medicine or treatment substances been suggested for treating such loss of hair and alopecia. For example, pills containing chemical substances have been introduced, or topical treatment products such as Rogaine, which is a trademark of Pfizer Inc. containing Minoxidil as an active ingredient for the treatment of male pattern hair loss, are commercially available. However, these known products have been known to be not effective in treating such symptoms or accompanying certain adverse side effects to the person using them.

Natural substances obtained from edible plants have been known to have various medical or cosmetic applications. For example, garlic (Allium sativum) has been known to be useful for lowering of LDL cholesterol levels and the inhibition of blood platelet clumping. For another example, onions (Allium cepa) have been known to lower blood pressure and help dissolve blood clots.

In particular, the use of materials derived from a plant of the Allium family, especially Allivum sativum (“garlic”) has been reported in the literature. For example, several patents by Tatarintsev et al report the use of ajoene compound, derived from the garlic plant, for treating a variety of ailments including AIDS, inflammation, arthritis, transplant, infection, autoimmune diseases such as lupus, tuberculosis, tumors, and other relates diseases. (See U.S. Pat. Nos. 5,856,363; 5,863,955; 5,948,821; and 5,932,621.) Also, Hibi, U.S. Pat. No. 5,612,077, describes an ajoene-containing extract from garlic for use in treating arteriosclerosis, tuberculosis, and bronchitis. Further, Tsuei, U.S. Pat. No. 4,795,636; Seebeck, U.S. Pat. No. 2,642,374; and Spinka et al, U.S. Pat. No. 2,618,561, describe garlic extracts as medicinal agents.

Further, the isolation of compounds from plant materials, including Allium that inhibit apoptosis or is useful for medical purposed has been reported. See especially, U.S. Pat. Nos. 6,340,483; 5,567,425; 5,759,548; 4,986,985; 5,620,885; 5,624,672; 5,635,186; and 5,635,187, by Bathurst et al, all of which are incorporated by reference in their entirety herein.

The foregoing is only exemplary of plant-derived materials reported to possess medicinal properties. However, notwithstanding the large number of plant-derived materials reported to possess therapeutic properties, there still exists a need for novel plant extracts and therapeutic use thereof as such therapies may be safer and more cost effective than traditional medical treatments.

Further, there still exists a need for providing natural hair/body care products, such as soap, shampoo, hair and body cleaning conditioner, topical composition for scalp, or the like, and compositions thereof, which do not contain harmful or toxic chemical substances and further function well particularly for cleaning and hair/skin care, along with added effect of reducing hair loss or stimulating hair growth.

### SUMMARY OF THE INVENTION

The present invention is particularly directed to topical compositions and hair/skin care products (e.g., soap, shampoos, rinses, and the like) containing chemical substances or plant components obtained from certain edible plants, in particular, plant species of the family Liliaceae (also known as Alliaceae) or Amaryllidaceae, preferably selected from the following Allium species in particular: Allium fistulosum (such as bunching onion, scallion, Welsh onion, green onion, or spring onion) and Allium ampeloprasum var. porrum (leek, wild leek, or Egyptian leek), which can stimulate hair growth and/or reduce hair loss. The topical compositions and products of the invention can also function well and safely without adverse effects for cleaning and caring hair, scalp and the body. The topical compositions and products may further include at least one ingredient selected from a group consisting of minerals, vitamin A, vitamin C, vitamin E, rosemary, lavender, thyme, yarrow, lemon, aloe vera, avocado, carrot seed, cabbage seed, ginger, ginseng, chamomile, birch, bay, cedarwood, grapefruit, and clary sage, which can also provide further effects for promoting hair growth and protecting or caring the skin.

The present invention is also directed to a method of making personal care products (such as soap, shampoos, rinses, and other kinds of hair or skin care products) which contain plant components or chemical substances obtained from certain edible plants, in particular, plant species of the family Liliaceae (also known as Alliaceae), preferably selected from the following Allium species: Allium fistulosum (bunching onion, scallion, Welsh onion, green onion, or spring onion) and Allium ampeloprasum var. porrum (leek, wild leek, or Egyptian leek).

The present invention is also directed to a method of promoting hair growth, the method comprising applying an effective amount of topical composition to the skin, the composition containing plant components or extracts obtained from at least one plant species of the family Liliaceae or Amaryllidaceae, the at least one plant species being selected from a species group consisting of Allium fistulosum, Allium ampeloprasum or Allium porrum, and other Allium species or their relative or equivalent species having ingredients which can stimulate hair growth or reduce hair loss.

### BRIEF DESCRIPTION OF DRAWINGS

FIG. 1 is a photographic view of bunching onions, Welsh or green onions (Allium fistulosum), extracts of which can be used for producing personal care products, such as soap, shampoos, rinses, or the like, according to the principles and concepts of the present invention.

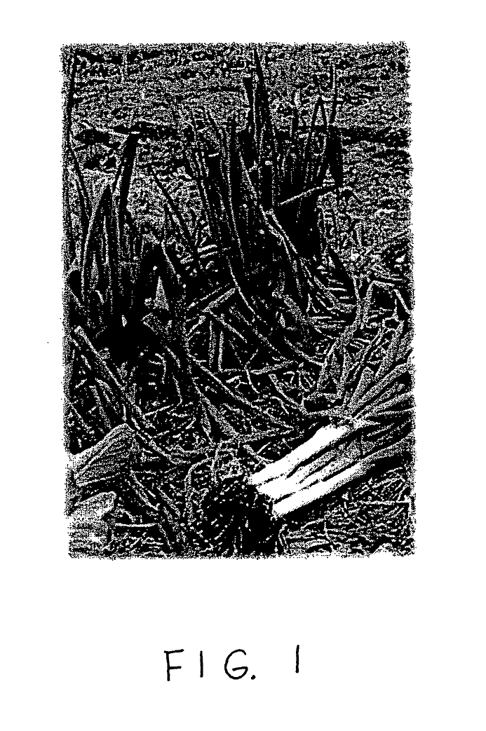

FIG. 2 is a flow chart for illustrating a general process to make soap with Allium fistulosum extracts or their equivalents, according to the invention.

FIG. 3 is a flow chart for illustrating a general process to make topical composition in liquid or solution with Allium fistulosum extracts or their equivalents, according to the invention.

### DETAILED DESCRIPTION OF PREFERRED EMBODIMENTS

The present invention is based on the discovery of this applicant that plants of the family Amaryllidaceae or Liliaceae, specifically those of Allium fistulosum, Allium ampeloprasum or Allium porrum, Allium cepa, and related species such as, Allium chinense G. Don, Allium schoenoprasum, and Allium ascalonicum can be used to obtain plant extracts having significant medicinal or therapeutic properties. In particular, the applicant has discovered that extracts derived from certain Allium species, preferably Allium fistulosum, Allium ampeloprasum or Allium porrum (however, excluding Allium sativum, i.e., garlic) have particularly useful medicinal or therapeutic properties, including activity against hair loss, activity stimulating hair cells for promoting hair growth to scalps, when topically applied.

In furtherance of this discovery, the present applicant of this invention had made several experiments with soap made by the applicant for test, which contains extracts from Allium fistulosum (such as bunching onions, Welsh onions or great green onions). The applicant had arranged to have a plurality of people with hair loss concerns use the soap regularly (e.g., once or twice daily) as experiments to test functioning of such plant extracts. More specifically, the soap was made by the applicant in a known method for making soap, however, added in the soap as active ingredients for reducing hair loss and/or for hair growth are extracts of Allium fistulosum, in particular, the plants called “Pa” or “Dae-Pa” (in Korean language) which are a relatively big and thick sort of bunching onions or great green onions available in Korean or Asian groceries (which may be similar to those called “Cong” or “Da cong” in Chinese language, and “Negi” in Japanese language). The test results have indicated that a substantial portion of people used this soap for a few months or so has reported that they found this soap to be helpful to prevent or reduce hair loss or even to promote hair growth after using the soap. The plant “Pa” or “Dae-Pa” is one of most favorite vegetables for Korean or other Asian foods. This “Pa” or “Dae-Pa” is believed by the applicant to be within the Allium fistulosum species in the family of Amaryllidaceae or Liliaceae. The plants “Pa” or “Dae-Pa” are also to be referred in this disclosure as “Korean or Asian great green onions”.

Applicant further believes other similar Allium species, such as Allium ampeloprasum or Allium porrum, might have similar functions as that of Allium fistulosum, however, Applicant believes Allium cepa (the common onion) and Allivum sativum (garlic) might be not as effective as Allium fistulosum, Allium ampeloprasum or Allium porrum. Applicant is planning to proceed with further researches in this regard.

Accordingly, the subject invention provides novel medicinal extracts for topical uses and methods of use thereof, wherein such medicinal extracts are derived from a plant of the family Amaryllidaceae or Liliaceae, more preferably of Allium fistulosum, Allium ampeloprasum (such as leeks), and Allium porrum, possibly of Allium cepa and Allium altaicum. Most preferably, the novel medicinal extracts of the present invention will be derived among other Allium species from Allium fistulosum (known as bunching onion, Japanese bunching onion, scallion, Welsh onion, green onion, spring onion, ciboule, or the like).

According to certain authorities (see for example http://www.floridata.com/ref/A/alli_fis.cfm), green onions to the cook, scallions at the super market, Welsh onions in England, ciboule in France, or bunching onions in most books, they all refer to Allium fistulosum, a very distinctive member of the onion family. Bunching onions form perennial evergreen clumps up to 1 ft (0.3 m) in diameter and about 2 ft (0.6 m) tall. The leaves are hollow and tube-like, inflated their entire length. The bulbs are elongate and not much thicker than the stem. After a cold spell, bunching onions send up hollow stalks topped with little greenish flowers in round umbels (clusters with all the flower stems arising from the same point), that are 1-3 in (2.5-7.6 cm) in diameter.

There are many cultivars, including some with red skins (‘Santa Clause’, ‘Red Beard’); some with shorter, thicker stalks (‘Shimonita’); some with larger bulbs (‘Yoshima’); and some that are exceptionally cold-hardy, such as ‘White Lisbon’, ‘Evergreen White Bunching’ and ‘Winter Over’. It is not unusual to harvest these under the snow. Bunching onions also have been hybridized with other Allium species, especially the common bulbing onion, A. cepa. These hybrids are sterile, and must be propagated by division of side shoots. ‘Beltsville Bunching’ is one such hybrid, noteworthy for its tolerance to hot and dry weather. The bunching onion was developed in Asia from a wild relative, possibly Allium altaicum, which occurs in NW China and neighboring Kazakhstan. It was brought to Europe in the 17th century.

Bunching onions are used extensively in oriental stir fries. They are also used raw in salads, as a garnish, and as a substitute for chives (Allium schoenoprasum) or leeks (Allium ampeloprasum, Porrum Group). Serving examples include an appetizer plate of scallions and other raw veggies such as radishes (Raphanus sativus), celery, kohlrabi (Brassica oleracea var. gongylodes), carrots (Daucus carota var. sativus), and broccoli (Brassica oleracea var. botrytis) with a creamy dip or just some salt.

Spring onions are small and immature bulbing onions (Allium cepa, Cepa Group), and are used in place of bunching onions. The name, green onions, can refer to either bunching onions or immature bulbing onions. The term scallion is used for bunching onions and also sometimes for shallots (a.k.a. multiplier onions, A. cepa, Aggregatum Group), which are a type of bulbing onion grown for their small garlic-like brownish bulbs, as well as for their leaves which are similar to those of bunching onion. The common name, Welsh onion, is derived from the German, “Walsch”, which means foreign, and has nothing at all to do with Wales.

Some authorities place the onions, garlics, leeks and their relatives in a family of their own, the Alliaceae, and others put them in the lily family, the Liliaceae. There are about 400 species in the genus Allium, including some magnificent ornamentals.

On the other hand, the Allium cepa species (i.e., the common bulbing onion) are divided into three main groups, according to U.S. Pat. No. 6,340,483, for example. The first group is the common onion—bulbs, which are formed as single plants, and the inflorescence does not form bulbets (Nonnecke, (Id.)1989). The bulk of the onion cultivars belong to this group, which is the most important commercially (Nonnecke, (Id.)1989). It is propagated in the main from true seeds. Within this group exist extremes of bulb shapes (present day cultivars include the Sweet Spanish, Bermuda, and globe onions), dry scale color (white, yellow, and red predominating), pungency (ranging from mild and sweet to pungent), and other characteristics (Nonnecke, (Id.)1989). When harvested early they produce spring or bunching onions (Lorenz, Onion, In “The Software Toolworks Multimedia Encyclopedia”, Version 1.5, Grolier, Inc. (1992)). The onion plant is potentially a biennial, producing large bulbs the first year and seed the next (Lorenz, (Id.)1992). Plants may be grown from seed, as transplants of seedlings, or as small bulbs (sets) produced from thickly planted seed; when replanted, these bulbs reach maturity rapidly (Lorenz, (Id.)1992). Mature onions are usually dried before marketing (Lorenz, (Id.)1992).

The second group (of the Allium cepa species) is the aggregatum group—characterized by many lateral bulbs or shoots, inflorescences lacking bulbets, sterile seed production, and propagation by vegetative means (Nonnecke, (Id.)1989). This group includes the potato onion or multiplier onion, ever-ready onions, and shallots (shallots are sometimes called scallions, a source of confusion because Allium fistulosum is also often called scallions) (Nonnecke, (Id.)1989). The third group is the proliferous (proliferum) group—in this group, ground bulbs are sometimes poorly developed, the inflorescence bears bulbets, true seed is usually lacking, and therefore reproduction is by vegetative inflorescence and bulbets (Nonnecke, (Id.)1989). These are not commercially cultivated; they are used almost exclusively for home gardening (Nonnecke, (Id.)1989). The most common names for this group are tree onions, top-set onions, and Egyptian onions (Nonnecke, (Id.)1989).

Several exemplary embodiments of the invention are described herein in particular in the form of several examples, which are to provide further guidance for illustrating, understanding and/or practicing the present invention. However, the following descriptions of the Examples are not intended to limit the scope of the present invention.

Example of Application: Making Soap

There are several known methods for making soap, including such as the Cold Process—the most common—making soap from scratch with oils, lye and other soap ingredients; the Melt and Pour Process—melt pre-made blocks of soap and add desired fragrance and other ingredients, which is relatively simple and even can be done at home; and other known commercial methods of soap making. The present invention can be applied to any known methods of soap making, including the ones specifically described herein.

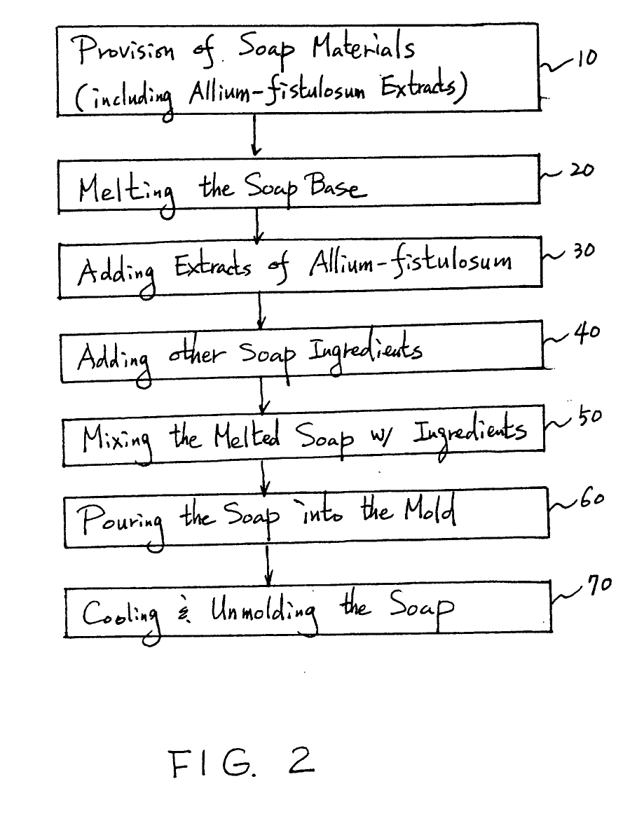

Referring to FIG. 2, one exemplary method of the soap of the invention is described herein, in particular, under the so-called Melt and Pour Process. However, as discussed, the present invention can be applied to any other known methods of soap making, such as the so-called Cold Process, etc.

First, soap materials are prepared (step 10). Soap materials include various known soap forming materials (for example, the “soap base” which is a pre-made block, when making the soap by the Melt and Pour Process) and additional ingredients such as essential oil, fragrance, colorants, etc.

In the soap material preparation process, extracts of Allium fistulosum are also prepared. One preferred example of the extracts of Allium fistulosum can be obtained from bunching onions, Welsh onions or great green onions, more preferably, from the plants “Pa” or “Dae-Pa” described above.

According to one preferred embodiment of the invention, selected plants of Allium fistulosum (bunching onions, Welsh onions, great green onions, or the like) of good condition, including the leaf and bulb portions optionally having root portions, are first cleaned. The cleaned plants are heated or steamed, and then dried to a desired degree under the sun or in the shade or subjecting to heat. More preferably, the steamed plants are roasted or parched with heat in order to fully and effectively dry. Strong odor of the Allium fistulosum plants can advantageously be eliminated through these processes of steaming and drying.

The dried plants are then processed in a crushing machine or mill into powder or particles of a predetermined or desired size. It is noted that extracts of Allium fistulosum or other similar Allium species can be obtained other known methods, except that making into powder form as described herein. For example, extracts can be obtained in the form of condensed juice or gel of the Allium plants after squeezing (in hot or cold condition) and processed in known methods. The condensed juice can be dried to a certain desired degree suitable for soap making.

Next, the “soap base” of precisely-measured amount is subject to heat and melted (step 20). For example, the soap base of 1 Kg weight is placed in a suitable container and melted completely in an electric or gas heater or in a Microwave heater.

Then, the extracts of Allium fistulosum or suitable other Allium species (in the form of dried powder as discussed, or other form of extracts known in the art) are added into the melting-soap container (step 30). According to one example, dried “Dae-Pa” of 50 gram weight is added to the soap base of 1 Kg weight previously placed in the melting container. This soap mixture is preferably stirred in order to adequately mix the plant extracts with the soap base.

Other soap ingredients, such as essential oil, fragrance, vitamin E, other active or inactive ingredients used for soap, and colorants, are added into the container (step 40). In the present invention, the applicant has also discovered certain minerals, vitamins (such as vitamin A, C, and E), and herbal or plant substances and essential oil are helpful for preventing or reducing hair loss or otherwise for stimulating hair growth to the scalp, while also providing proper care to the skin and hair. Such herbal substances and/or essential oil of the invention include, without limitation thereto, minerals, vitamins, rosemary, lavender, thyme, yarrow, lemon, aloe, avocado, ginger, carrot seed, cabbage seed, ginseng, chamomile, birch, bay, cedarwood, grapefruit, clary sage, etc.

Then, the melted soap base with the extracts of the Allium species and other ingredients is stirred or mixed adequately before it is solidified by cooling in the container (step 50).

The hot soap mixture is then poured into a mold for making bars of desired shapes (step 60).

The soap is subject to a cooling process to cool it completely, preferably in a cooler or refrigerator, and the completed bars of soap are pulling from the mold (step 70). The soap may be subject to a further drying process before using.

Examples of other Applications: Making Shampoo, Hair Conditioner, Rinse, or Other Liquified Matter

The topical composition of the invention obtained from extracts from the above-specified Allium species (preferably Allium fistulosum or other Allium species of similar property) can be used as active ingredients for making other hair-care related products, including shampoos, hair conditioners or rinses, or other liquefied matters for topical application to the scalp and hair. For these applications, the plant extracts can be made in powder form as described above, or more preferably, they can be made in liquefied or gel-like form or in solution with other known ingredients of the products.

General ingredients of shampoos, hair conditioners, or the like, and methods for making the same are well known in the art. The present invention can utilize such known ingredients and methods of making such hair care products, and thus, they are not discussed herein in details. Accordingly, descriptions of the present invention are focused on making the plant extracts.

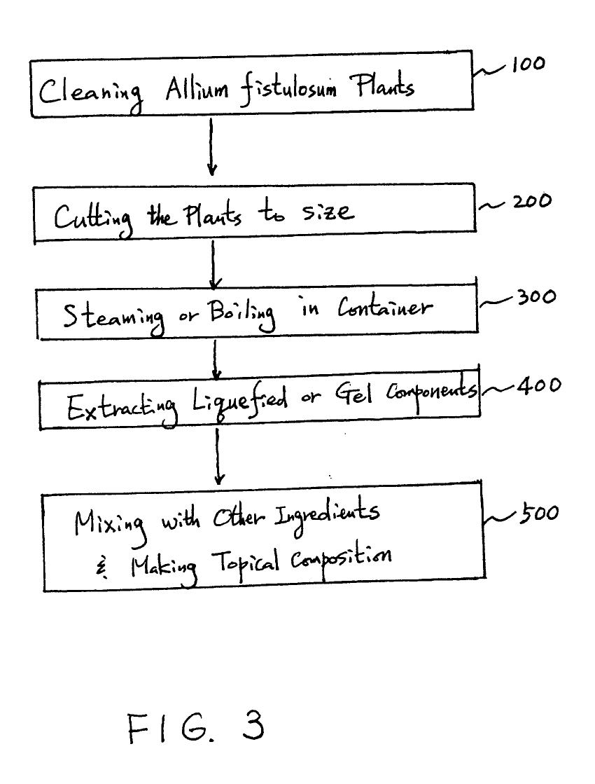

Exemplary methods for making the topical composition or plant extracts in the form of liquid, solution, gel, or their mixture are further described herein, with reference to FIG. 3.

According to one preferred embodiment of the invention, bunching onions, Welsh onions, great green onions, or Korean “Pa” or “Dae-Pa” (also referred as “Korean or Asian great green onions”), or the like, of good condition are selected and cleaned by known methods (step 100). The cleaned bunching onions may be subject to cutting to a predetermined size (step 200). They are then put in a suitable container, and are subject to heat, steamed or boiled with water (step 300).

The heated bunching onions (preferably the whole plants with or without the roots) are subject to an extraction process for extracting liquefied or gel components of active ingredient of the plants from the plants in a method known in the art (step 400). For example, the plants (preferably cut in small size or particles) are squeezed in a squeezing machine to obtain active components of the plant by squeezing while removing inactive components such as cellulose or other structural components from the extracts.

The plant extracts are stored in a cool temperature, preferably in a refrigerator for future use to make the ultimate products of desire. Otherwise, when it's ready for further processes, they are mixed with other ingredients of the topical compositions as known in the art for making the hair care products (e.g., shampoo or the like) by mixing and processing in a conventional or known method (step 500).

As discussed above, the present applicant has further discovered that, the following ingredients (in form of either oil or extracts) are also helpful, in addition to the Allium plant extracts described above, for preventing or reducing hair loss or for stimulating hair growth to the scalp, while also providing proper care to the skin and hair: such as minerals, vitamin A, vitamin C, vitamin E, rosemary, lavender, thyme, yarrow, lemon, aloe (e.g., aloe vera), avocado, carrot seed, ginger, ginseng, chamomile, birch, bay, cedarwood, grapefruit, clary sage, etc. In addition, a wide variety of optional ingredients such as non-occlusive moisturizers, humectants, gelling agents, neutralizing agents, perfumes, coloring agents and surfactants can be added to the presently contemplated topical compositions.

According to the invention as described above with exemplary embodiments thereof, the topical compositions and hair/body care products are formed essentially with natural and non-toxic ingredients such as extracts from the plants of Allium species group, which can function effectively and safely for cleaning the hair, scalp, and the body, while providing additional effects for stimulating hair growth or for preventing or reducing hair loss. Application of the topical composition to the skin or scalp for promoting hair growth or reducing hair loss is also contemplated by this invention, for example, as exemplified in the accompanied claims of this application.

While this invention has been particularly illustrated and described with reference to preferred or exemplary embodiments thereof, it will be understood by those skilled in the art that various changes in form and details may be made therein without departing from the spirit and scope of the invention, for example, as exemplified in the claims accompanied herein.

What is claimed is:

1. A topical composition for a beauty care product, comprising plant components obtained from at least one plant species of the family Liliaceae or Amaryllidaceae, the at least one plant species being selected from a species group consisting of Allium fistulosum, Allium ampeloprasum or Allium porrum, and other Allium species or their relative or equivalent species having ingredients which can stimulate hair growth or reduce hair loss, the topical composition having a further property for a beauty care.

2. The topical composition of claim 1, wherein the at least one plant species is selected from bunching onion, scallion, Welsh onion, green onion, great green onion, spring onion, leek, wild leek, Egyptian leek, shallot, and Korean or Asian great green onion.

3. The topical composition of claim 1, wherein the topical composition is usable for soap, shampoos, rinses, body conditioners, hair conditioners, and the like.

4. The topical composition of claim 1, further comprising a property of cleaning or cleansing hair, scalp, and a body.

5. The topical composition of claim 4, wherein the topical composition has a reduced odor of the plant species selected.

6. The topical composition of claim 1, wherein the plant species is Allium fistulosum.

7. The topical composition of claim 1, further comprising at least one ingredient selected from a group consisting of minerals, vitamin A, vitamin C, vitamin E, rosemary, lavender, thyme, yarrow, lemon, aloe vera, avocado, carrot seed, cabbage seed, ginger, ginseng, chamomile, birch, bay, cedarwood, grapefruit, and clary sage.

8. The topical composition of claim 7, further comprising at least one further ingredient selected from a group consisting of non-occlusive moisturizers, humectants, cleaning agents, softening agents, gelling agents, neutralizing agents, perfumes, coloring agents, and surfactants.

9. The topical composition of claim 1, wherein the plant components are in the form of plant extracts.

10. The topical composition of claim 9, wherein the plant extracts are generally in powder, granular or particle form.

11. The topical composition of claim 9, wherein the plant extracts are generally in liquefied, solution, or gel-like form.

12. Soap or a cleaning substance, comprising plant components obtained from at least one plant species of the family Liliaceae or Amaryllidaceae, the plant species selected from a species group consisting of Allium fistulosum, Allium ampeloprasum or Allium porrum, and other Allium species or their relative or equivalent species having ingredients, which can stimulate hair growth or reduce hair loss.

13. The soap or cleaning substance of claim 12, wherein the at least one plant species is selected from bunching onion, scallion, Welsh onion, green onion, great green onion, spring onion, leek, wild leek, Egyptian leek, shallot, and Korean or Asian great green onion.

14. The soap or cleaning substance of claim 13, further comprising at least one ingredient selected from a group consisting of minerals, vitamin A, vitamin C, vitamin E, rosemary, lavender, thyme, yarrow, lemon, aloe vera, avocado, carrot seed, cabbage seed, ginger, ginseng, chamomile, birch, bay, cedarwood, grapefruit, and clary sage.

15. A method of making a personal beauty care product, the personal beauty care product containing plant components obtained from at least one plant species of the family Liliaceae or Amaryllidaceae, the method comprising:
selecting at least one plant species from a species group consisting of Allium fistulosum, Allium ampeloprasum or Allium porrum, and other Allium species or their relative or equivalent species having ingredients which can stimulate hair growth or reduce hair loss; and
extracting active ingredients of the plant species for stimulating hair growth or reducing hair loss.

16. The method of claim 15, wherein said extracting of active ingredients of the plant species is performed in the form of powder, granules, or particles.

17. The method of claim 15, wherein said extracting of active ingredients of the plant species is performed in the form of liquid, solution, gel, and their mixture.

18. The method of claim 15, further comprising cutting the selected plants to size before said extracting.

19. The method of claim 18, further comprising steaming or boiling the selected plants before said extracting.

20. The method of claim 15, further comprising roasting, parching, or heating the selected plants before said extracting.

21. The method of claim 15, further comprising drying the selected plants before said extracting.

22. The method of claim 15, further comprising adding ingredients for cleaning or cleansing.

23. A method of promoting hair growth comprising applying an effective amount of topical composition to the skin, the composition containing plant components obtained from at least one plant species of the family Liliaceae or Amaryllidaceae, the at least one plant species being selected from a species group consisting of Allium fistulosum, Allium ampeloprasum or Allium porrum, and other Allium species or their relative or equivalent species having ingredients which can stimulate hair growth or reduce hair loss.

24. The method of claim 23, wherein the species are selected from Allium fistulosum.
